SEQUENCE LISTING (3 sequences)

SEQ ID NO: 1 - protein, 626 aa, synthetic construct
  source 1..626: note = Amino acid sequence encoded by mRNA-1
MDAMKRGLCCVLLLCGAVFVSPSEAAAKEAAAKMWNLLHETDSAVATARRPRWVLAGGFFLLKKFLYNFTQIPHLAGTGGGGSGGGGSGGGGSNVSDIVPPFSAFSPQGMPEGDLVYVNYARTEDFFKLEGGGGSGGGGSGGGGSARYGKVFRGNKVKNAQLAGAKGVILYSDPADYFAPGGGGGSGGGGSGGGGSSRLLQERGVAYINADSSIEGNYTLRVDCTPLMYSLVHNLTKEGGGGSGGGGSGGGGSFYDPMFKYHLVLRKYADKIYSISMKHPQEMKTYSVSFDSLFSAVGGGGSGGGGSGGGGSVLRMMNDQLMFLSHNKYAGESFPGIYDALFDIESKVDPSGGGGSGGGGSGGGGSAGLALQPGTALLCYSNCTQLGEQCWTARIRGGGGSGGGGSGGGGSDTDLCNASGAHALQPAAAILALLPALGLLLWGPGQLGGGGSGGGGSGGGGSPQWHLPIKIAAIIASLTFLYTLLREVIHPLATSHQQYFYGGGGSGGGGSGGGGSLVYLPGVIAAIVQLLTRKQFGLLSFFFAVLHAIYSLSYPGGGGSGGGGSGGGGSIVGIVAGLAVLAVLAVLGAMVAVVMCRRKSSGGKGGSCSQAASSNSAQGSDVSLTA

SEQ ID NO: 2 - protein, 704 aa, synthetic construct
  source 1..704: note = Amino acid sequence encoded by mRNA-2
MDAMKRGLCCVLLLCGAVFVSPAKFVAAWTLKAAAMWNLLHETDSAVATARRPRWVLAGGFFLLKKFLYNFTQIPHLAGTGGGGSGGGGSGGGGSNVSDIVPPFSAFSPQGMPEGDLVYVNYARTEDFFKLEGGGGSGGGGSGGGGSARYGKVFRGNKVKNAQLAGAKGVILYSDPADYFAPGGGGGSGGGGSGGGGSSRLLQERGVAYINADSSIEGNYTLRVDCTPLMYSLVHNLTKEGGGGSGGGGSGGGGSFYDPMFKYHLVLRKYADKIYSISMKHPQEMKTYSVSFDSLFSAVGGGGSGGGGSGGGGSVLRMMNDQLMFLSHNKYAGESFPGIYDALFDIESKVDPSGGGGSGGGGSGGGGSAGLALQPGTALLCYSNCTQLGEQCWTARIRGGGGSGGGGSGGGGSDTDLCNASGAHALQPAAAILALLPALGLLLWGPGQLGGGGSGGGGSGGGGSPQWHLPIKIAAIIASLTFLYTLLREVIHPLATSHQQYFYGGGGSGGGGSGGGGSLVYLPGVIAAIVQLLTRKQFGLLSFFFAVLHAIYSLSYPGGGGSGGGGSGGGGSSQSTIPIVGIVAGLAVLAVVVIGAVVATVMCRRKSSGGKGGSYSQAASSDSAQGSDVSLTAGGGGSGGGGSGGGGSIVGIVAGLAVLAVLAVLGAMVAVVMCRRKSSGGKGGSCSQAASSNSAQGSDVSLTA

SEQ ID NO: 3 - protein, 810 aa, synthetic construct
  source 1..810: note = Amino acid sequence encoded by mRNA-3
MRVTAPRTVLLLLSAALALTETWAAKFVAAWTLKAAAMWNLLHETDSAVATARRPRWVLAGGFFLLKKFLYNFTQIPHLAGTGGGGSGGGGSGGGGSNVSDIVPPFSAFSPQGMPEGDLVYVNYARTEDFFKLEGGGGSGGGGSGGGGSARYGKVFRGNKVKNAQLAGAKGVILYSDPADYFAPGGGGGSGGGGSGGGGSSRLLQERGVAYINADSSIEGNYTLRVDCTPLMYSLVHNLTKEGGGGSGGGGSGGGGSFYDPMFKYHLVLRKYADKIYSISMKHPQEMKTYSVSFDSLFSAVGGGGSGGGGSGGGGSVLRMMNDQLMFLSHNKYAGESFPGIYDALFDIESKVDPSGGGGSGGGGSGGGGSAGLALQPGTALLCYSNCTQLGEQCWTARIRGGGGSGGGGSGGGGSDTDLCNASGAHALQPAAAILALLPALGLLLWGPGQLGGGGSGGGGSGGGGSPQWHLPIKIAAIIASLTFLYTLLREVIHPLATSHQQYFYGGGGSGGGGSGGGGSLVYLPGVIAAIVQLLTRKQFGLLSFFFAVLHAIYSLSYPGGGGSGGGGSGGGGSSQSTIPIVGIVAGLAVLAVVVIGAVVATVMCRRKSSGGKGGSYSQAASSDSAQGSDVSLTAGSGATNFSLLKQAGDVEENPGPMYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2026/0166134 A1
(43) Pub. Date: Jun. 18, 2026

(54) mRNA, mRNA VACCINE FORMULATION, AND PREPARATION METHOD AND USE THEREOF

(71) Applicant: Panru Biotechnology (Tianjin) Co., Ltd, Tianjin (CN)

(72) Inventors: Yuanjie NIU, Tianjin (CN); Jianpeng YU, Tianjin (CN); Keruo WANG, Tianjin (CN); Peng LIU, Tianjin (CN); Yuejia NIU, Tianjin (CN)

(73) Assignee: Panru Biotechnology (Tianjin) Co., Ltd, Tianjin (CN)

(21) Appl. No.: 19/258,958

(22) Filed: Jul. 3, 2025

(30) Foreign Application Priority Data
Dec. 12, 2024 (CN) 202411823089.1

(51) Int. Cl. A61K 39/00; A61P 37/04

(52) U.S. Cl. CPC A61K 39/001193; A61P 37/04; A61K 2039/53; A61K 2039/6093; A61K 2039/884

(57) ABSTRACT

An mRNA, an mRNA vaccine formulation, and a preparation method and use thereof are provided. An amino acid sequence of the mRNA is set forth in SEQ ID NO: 2. Studies have demonstrated that the mRNA vaccine formulation prepared from the mRNA having the amino acid sequence set forth in SEQ ID NO: 2 can effectively stimulate dendritic cell maturation and secretion of pro-inflammatory cytokines, thereby activating effector T cells to kill prostate cancer cells. A preparation method of the mRNA vaccine formulation is simple and suitable for industrial-scale production. By using lipid nanoparticles (LNPs) as a carrier, the mRNA vaccine formulation achieves high encapsulation efficiency, enabling robust induction of immune responses in vivo with high immunogenicity.

### CROSS REFERENCE TO RELATED APPLICATION

This application is based upon and claims priority to Chinese Patent Application No. 202411823089.1, filed on Dec. 12, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0257_SequenceListing.xml, created on 06/27/2025, and is 6,198 bytes in size.

### TECHNICAL FIELD

The present disclosure belongs to the technical field of biomedicine, and in particular relates to a messenger ribonucleic acid (mRNA), an mRNA vaccine formulation, and a preparation method and use thereof.

### BACKGROUND

Prostate cancer ranks among the cancers with the highest incidence and mortality rates in men globally. In the United States, prostate cancer has become the most severe malignant tumor threatening male health. With accelerating population aging and increased consumption of Westernized diets, the incidence of prostate cancer has surged in recent years, emerging as a major killer endangering the health and quality of life of elderly males. Early-stage prostate cancer lacks specific clinical symptoms and progresses insidiously, generally reaching intermediate or advanced stages by the time noticeable symptoms appear. Current treatments for early-stage prostate cancer cases include radical prostatectomy, androgen deprivation therapy (ADT), and radiotherapy, but patients may still experience recurrence after definitive localized therapies. Moreover, prostate cancer exhibits a high propensity for bone metastasis, and most patients clinically lose eligibility for curative surgery at initial diagnosis. For advanced prostate cancer, chemotherapy and endocrine therapy are commonly used, but their clinical utility is limited by issues such as patient intolerance to chemotherapy side effects and endocrine therapy resistance. Consequently, therapeutic tumor vaccines show great promise as adjuvant therapies to prevent recurrence post-radical prostatectomy in early-stage cases, and as treatments for metastatic hormone-sensitive prostate cancer (mHSPC) and metastatic castration-resistant prostate cancer (mCRPC) in advanced stages where surgery is no longer feasible.

A successful cancer vaccine must induce the recruitment of antigen-presenting cells (APCs), typically dendritic cells and macrophages. After antigen uptake and activation, APCs transport the antigens to draining lymph nodes to activate and expand antigen-specific T cells. These T cells must then exit lymphoid organs, migrate into the tumor microenvironment (TME), and exert effector functions. Upon entering the TME, T cells face multiple challenges, including stromal architecture, which confines many tumor-infiltrating lymphocytes to perivascular regions rather than the tumor interior. Additionally, T cells encounter physical and chemical barriers within the TME, such as stromal cells, tumor cells, and immunosuppressive cells (e.g., myeloid-derived suppressor cells, tumor-associated macrophages, and regulatory T cells) suppress cytotoxic T cell activity by releasing immunomodulatory cytokines (e.g., TGF-β and IL-10) and expressing immune checkpoint molecules and ligands (e.g., PD-1/PD-L1). Furthermore, immunosuppressive cells can release immune regulatory metabolites like indoleamine 2,3-dioxygenase (IDO), which metabolizes tryptophan essential for T cell survival into kynurenine, further inhibiting activity of T cell. A successful cancer vaccine must rapidly amplify its effects to overcome these barriers and generate an antitumor immune response capable of inhibiting cancer progression.

mRNA vaccines demonstrated significant value during the COVID-19 pandemic due to their rapid development and efficacy in preventing SARS-CoV-2 infection, and this technology holds considerable potential for cancer treatment. Compared to traditional protein- or peptide-based antitumor vaccines, mRNA vaccines are not restricted by patient-specific human leukocyte antigen (HLA) haplotypes. They can be engineered to enhance stability while preserving immunogenicity and are capable of inducing complementary innate and adaptive immune responses. To date, although some Phase I/II clinical trials have yielded promising results, no mRNA tumor vaccine has yet received regulatory approval for clinical therapeutic use.

To enhance mRNA vaccine production speed, protect ribonucleic acid (RNA) from degradation in vivo, and accelerate uptake of mRNA vaccine by antigen-presenting cells, lipid nanoparticle (LNP)-based carrier systems have been widely adopted for mRNA vaccine manufacturing. LNPs are typically composed of ionizable lipids, helper lipids, cholesterol, and polyethylene glycol (PEG). With diameters usually below 200 nm, LNPs facilitate cellular endocytosis while ensuring payload release before endosome-lysosome fusion and subsequent degradation. LNPs typically include the ionizable lipids to encapsulate negatively charged mRNA molecules, as well as helper lipids to promote efficient payload delivery and endosomal escape into the cytoplasm. Cholesterol is usually added to LNPs to improve lipid membrane fluidity and transfection efficiency, and PEG is added to prolong nanoparticle stability and circulation time. Iterations of these technologies have been or are being applied to cancer vaccine development. Conventional in vitro-transcribed mRNA consists of a 5′ cap, 5′ and 3′ untranslated regions (UTRs), an open reading frame (ORF), and a polyadenylation (Poly(A)) tail. Such artificially transcribed mRNA is highly customizable, simple to produce, and scalable for mass production to treat diverse diseases. Unlike DNA-based therapies, mRNA does not require nuclear entry, eliminating risks of genetic material alteration. However, reducing immunogenicity and improving protein expression efficiency remain critical for mRNA-based therapies. Structural optimizations can enhance mRNA stability and translation efficiency. For example, modifying the 5′ terminus with autologous or synthetic 5′ cap analogs can increase stability and translation efficiency while modulating immune responses.

Previous Phase I/II clinical trials have demonstrated that CV9103, an mRNA vaccine encoding four prostate tumor-associated antigens (PSA, PSMA, PSCA, and STEAP1), can exhibit favorable tolerability and robust immune activation in prostate cancer treatment. However, subsequent clinical trials of CV9104, which includes two additional antigens (PAP and Mucin-1), are discontinued due to a lack of improvement in overall survival of patient. These findings indicate the critical importance of antigen selection in activating APCs and driving effective immune responses. Furthermore, as discussed earlier, the selection of LNP species, the modification of LNP to target APC cells and the optimization of mRNA backbone structures are pivotal for developing transformative cancer mRNA vaccines with clinical potential.

### SUMMARY

In view of the foregoing, an objective of the present disclosure is to provide an mRNA, an mRNA vaccine formulation, and a preparation method and use thereof. The mRNA vaccine formulation, prepared from the mRNA having the amino acid sequence set forth in SEQ ID NO: 2, effectively stimulates bone marrow-derived dendritic cells (BMDCs) maturation and secretion of pro-inflammatory cytokines, thereby inducing T lymphocyte-mediated killing of prostate cancer cells.

To achieve the above objective, the present disclosure provides the following technical solutions:

The present disclosure provides an mRNA having the amino acid sequence set forth in SEQ ID NO: 2.

The present disclosure further provides an mRNA vaccine formulation, including the mRNA.

In some embodiments, a carrier for the mRNA vaccine formulation is a lipid nanoparticle (LNP).

The present disclosure further provides a preparation method of an mRNA vaccine formulation, including the following steps:
diluting the mRNA to obtain an aqueous phase solution;
mixing 9-heptadecanyl 8-{(2-hydroxyethyl)[6-oxo-6-(undecyloxy)hexyl]amino}octanoate (SM-102), 1,2-distearoyl-sn-glycero-3-phosphocholine (DSPC), cholesterol, and 1,2-dimyristoyl-rac-glycero-3-methoxypolyethylene glycol 2000 (DMG-PEG2000) to obtain a lipid phase solution; and
combining the aqueous phase solution and the lipid phase solution to obtain the mRNA vaccine formulation.

In some embodiments, the mRNA is diluted to a concentration of 90 μg/mL to 95 μg/mL; the SM-102, the DSPC, the cholesterol, and the DMG-PEG2000 are mixed at a molar ratio of (45-55):(9-11):(35-40):(1-2); and the aqueous phase solution and the lipid phase solution are combined at a volume ratio of (1-6):(1-3).

The present disclosure further provides use of the mRNA, the mRNA vaccine formulation, or an mRNA vaccine formulation prepared by the preparation method in preparing a dendritic cell that is mature and/or secretes a pro-inflammatory cytokine.

The present disclosure further provides use of the mRNA, the mRNA vaccine formulation, or an mRNA vaccine formulation prepared by the preparation method in preparing a drug for treating prostate cancer.

The present disclosure further provides a method for preparing a dendritic cell that is mature and/or secretes a pro-inflammatory cytokine, including co-incubating a native dendritic cell with the mRNA vaccine formulation or an mRNA vaccine formulation prepared by the preparation method.

The present disclosure further provides a method for preparing an effector T cell, including co-incubating the dendritic cell prepared by the above method with a T lymphocyte.

The present disclosure further provides use of the effector T cell prepared by the above method in preparing a drug for treating prostate cancer.

Compared with the prior art, the present disclosure has the following beneficial effects:

The present disclosure provides an mRNA, an mRNA vaccine formulation, and a preparation method and use. Studies have demonstrated that the mRNA vaccine formulation prepared from the mRNA having the amino acid sequence set forth in SEQ ID NO: 2 can effectively stimulate dendritic cell maturation and secretion of pro-inflammatory cytokines, thereby activating effector T cells to kill prostate cancer cells. A preparation method of the mRNA vaccine formulation herein is simple and suitable for industrial-scale production. By using LNPs as a carrier, the mRNA vaccine formulation achieves high encapsulation efficiency, enabling robust induction of immune responses in vivo with strong targeting and desirable anti-tumor activity.

### BRIEF DESCRIPTION OF THE DRAWINGS

FIG. 1A shows the percentage of CD80⁺CD86⁺ BMDCs after stimulation with different mRNA vaccines; FIG. 1B shows TNF-α secretion levels in BMDCs stimulated by the different mRNA vaccines;

FIG. 2 shows the activation of T cells by BMDCs stimulated with different mRNA vaccines and the subsequent killing efficiency of these T cells against Tramp C1 target cells;

FIG. 3 shows the proportion of CD80⁺CD86⁺ monocyte-derived dendritic cells (MoDCs) after stimulation with different mRNA vaccines;

FIG. 4 shows the experimental protocol for treating the Tramp C1 subcutaneous tumor model of prostate cancer in mice with mRNA vaccines;

FIG. 5A shows photographs of Tramp C1 subcutaneous tumor on day 40 in prostate cancer post-treatment across different treatment groups; FIG. 5B shows weights of Tramp C1 subcutaneous tumor on day 40 in prostate cancer post-treatment across different treatment groups;

FIG. 5C shows tumor growth curves post-treatment across different treatment groups; FIG. 5D shows schematic diagram of mouse body weight post-treatment across different treatment groups; and

FIG. 6A shows the proportion of infiltrating T lymphocytes in the spleens of C57BL/6 mice post-treatment across different treatment groups; FIG. 6B shows the percentage of CD3⁺CD44^{high}CD62L^{high} central memory T lymphocytes post-treatment across different treatment groups.

### DETAILED DESCRIPTION OF THE EMBODIMENTS

The present disclosure provides an mRNA having the amino acid sequence set forth in SEQ ID NO: 2.

In the present disclosure, three distinct mRNA vaccines are prepared using mRNA constructs encoding three different amino acid sequences. Studies have revealed that the mRNA vaccine formulation derived from the mRNA having the amino acid sequence set forth in SEQ ID NO: 2 efficiently stimulates robust immune responses in vivo, exhibits strong target specificity, and demonstrates superior antitumor activity.

The present disclosure further provides use of the mRNA having the amino acid sequence set forth in SEQ ID NO: 2 in preparing an mRNA vaccine formulation.

The present disclosure further provides an mRNA vaccine formulation, including the mRNA.

In the present disclosure, the mRNA vaccine formulation employs an LNP as a carrier. The LNP includes SM-102, DSPC, cholesterol, and DMG-PEG2000. In some embodiments, the SM-102, the DSPC, the cholesterol, and the DMG-PEG2000 are mixed at a molar ratio of (45-55):(9-11):(35-40):(1-2). In some embodiments, the SM-102, the DSPC, the cholesterol, and the DMG-PEG2000 are mixed at a molar ratio of (48-52):(9.5-10.5):(37-39):(1.2-1.8). In some embodiments the SM-102, the DSPC, the cholesterol, and the DMG-PEG2000 are mixed at a molar ratio of 50:10:38.5:1.5.

The present disclosure further provides a preparation method of an mRNA vaccine formulation, including the following steps:
diluting the mRNA to obtain an aqueous phase solution;
mixing SM-102, DSPC, cholesterol, and DMG-PEG2000 to obtain a lipid phase solution; and
combining the aqueous phase solution and the lipid phase solution to obtain the mRNA vaccine formulation.

In the present disclosure, the mRNA is diluted to obtain an aqueous phase solution. The mRNA is diluted to a concentration of 90-95 μg/mL. In some embodiments, the mRNA is diluted to a concentration of 90.5-93 μg/mL. In some embodiments, the mRNA is diluted to a concentration of 91.75 μg/mL. The diluting involves first diluting the mRNA with water for injection to 180-185 μg/mL, followed by further dilution to 90-95 μg/mL using a citrate buffer (pH=4.0, (90-110) mM). The citrate buffer (pH=4.0) is purchased from Nanjing Chemical Reagent Co., Ltd. (Catalog No. C0132125124). In some embodiments, the SM-102, the DSPC, the cholesterol, and the DMG-PEG2000 are mixed at a molar ratio of (45-55):(9-11):(35-40):(1-2). In some embodiments, the SM-102, the DSPC, the cholesterol, and the DMG-PEG2000 are mixed at a molar ratio of (48-52):(9.5-10.5):(37-39):(1.2-1.8). In some embodiments, the SM-102, the DSPC, the cholesterol, and the DMG-PEG2000 are mixed at a molar ratio of 50:10:38.5:1.5. SM-102 is purchased from Beijing Jenkem Technology Co., Ltd. (Catalog No. S003), DSPC is purchased from Beijing Jenkem Technology Co., Ltd. (Catalog No. S038), and DMG-PEG2000 is purchased from the Beijing Jenkem Technology Co., Ltd. (Catalog No. A3294). In some embodiments, the aqueous phase solution and lipid phase solution are combined at a volume ratio of (1-6):(1-3). The formulation is homogenized using a microfluidic device under the following parameters: N/P ratio=6:1, aqueous phase flow rate: ethanol phase flow rate=3:1, and total flow rate of 12 mL/min. In the present disclosure, the lipid is dissolved in ethanol (as an organic solvent), hence a resulting ethanol phase is the lipid phase.

The present disclosure further provides use of the mRNA, the mRNA vaccine formulation, or an mRNA vaccine formulation prepared by the preparation method in preparing a dendritic cell that is mature and/or secretes a pro-inflammatory cytokine.

In the present disclosure, the dendritic cell includes a bone marrow-derived dendritic cell (BMDC) and/or a human peripheral blood mononuclear cell-derived dendritic cell (MoDC).

The present disclosure further provides use of the mRNA, the mRNA vaccine formulation, or an mRNA vaccine formulation prepared by the preparation method in preparing a drug for treating prostate cancer.

The present disclosure further provides a method for preparing a dendritic cell that is mature and/or secretes a pro-inflammatory cytokine, including co-incubating a native dendritic cell with the mRNA vaccine formulation or an mRNA vaccine formulation prepared by the preparation method.

In the present disclosure, the co-incubating is conducted for 20 h to 26 h (e.g., 22 h or 24 h). The mRNA vaccine formulation is used at a concentration of 8-12 μg/mL; and the dendritic cell, when being used in a six-well plate, is at a density of 0.5×10⁶ to 1.5×10⁶ cells/well.

The present disclosure further provides a method for preparing an effector T cell, including co-incubating the dendritic cell prepared by the above method with a T lymphocyte.

In the present disclosure, the ratio of dendritic cell to T lymphocyte is 1:8 to 1:12 (e.g., 1:10).

The present disclosure further provides use of the effector T cell prepared by the above method in preparing a drug for treating prostate cancer.

Studies herein demonstrate that the effector T cell are capable of kill prostate cancer cells efficiently.

The technical solutions provided by the present disclosure will be described in detail below with reference to examples, but the examples should not be construed as limiting the claimed scope of the present disclosure.

In the following examples, the plasmid DNA (pDNA) is purchased from Jiangsu Synthgene Biotechnology Co., Ltd. Water for injection (WFI) is purchased from Shanghai LePure Biotech (Catalog No. RWO20-02). The restriction enzyme buffer is purchased from Yeasen Biotechnology (Catalog No. 10664ES92). The CAP analog is purchased from Jiangsu Synthgene Biotechnology Co., Ltd. (Catalog No. CAP3011).

### Example 1

In this example, three mRNA constructs (designated mRNA-1, mRNA-2, and mRNA-3) were designed. The amino acid sequence of mRNA-1 was as follows:

(SEQ ID NO: 1)
MDAMKRGLCCVLLLCGAVFVSPSEAAAKEAAAKMWNLLHETDSAVATARR
PRWVLAGGFFLLKKFLYNFTQIPHLAGTGGGGSGGGGSGGGGSNVSDIVP
PFSAFSPQGMPEGDLVYVNYARTEDFFKLEGGGGSGGGGSGGGGSARYGK
VFRGNKVKNAQLAGAKGVILYSDPADYFAPGGGGGSGGGGSGGGGSSRLL
QERGVAYINADSSIEGNYTLRVDCTPLMYSLVHNLTKEGGGGSGGGGSGG
GGSFYDPMFKYHLVLRKYADKIYSISMKHPQEMKTYSVSFDSLFSAVGGG
GSGGGGSGGGGSVLRMMNDQLMFLSHNKYAGESFPGIYDALFDIESKVDP
SGGGGSGGGGSGGGGSAGLALQPGTALLCYSNCTQLGEQCWTARIRGGGG
SGGGGSGGGGSDTDLCNASGAHALQPAAAILALLPALGLLLWGPGQLGGG
GSGGGGSGGGGSPQWHLPIKIAAIIASLTFLYTLLREVIHPLATSHQQYF
YGGGGSGGGGSGGGGSLVYLPGVIAAIVQLLTRKQFGLLSFFFAVLHAIY
SLSYPGGGGSGGGGSGGGGSIVGIVAGLAVLAVLAVLGAMVAVVMCRRKS
SGGKGGSCSQAASSNSAQGSDVSLTA.

The amino acid sequence of mRNA-2 was as follows:

(SEQ ID NO: 2)
MDAMKRGLCCVLLLCGAVFVSPAKFVAAWTLKAAAMWNLLHETDSAVATA
RRPRWVLAGGFFLLKKFLYNFTQIPHLAGTGGGGSGGGGSGGGGSNVSDI
VPPFSAFSPQGMPEGDLVYVNYARTEDFFKLEGGGGSGGGGSGGGGSARY
GKVFRGNKVKNAQLAGAKGVILYSDPADYFAPGGGGGSGGGGSGGGGSSR
LLQERGVAYINADSSIEGNYTLRVDCTPLMYSLVHNLTKEGGGGSGGGGS
GGGGSFYDPMFKYHLVLRKYADKIYSISMKHPQEMKTYSVSFDSLFSAVG
GGGSGGGGSGGGGSVLRMMNDQLMFLSHNKYAGESFPGIYDALFDIESKV
DPSGGGGSGGGGSGGGGSAGLALQPGTALLCYSNCTQLGEQCWTARIRGG
GGSGGGGSGGGGSDTDLCNASGAHALQPAAAILALLPALGLLLWGPGQLG
GGGSGGGGSGGGGSPQWHLPIKIAAIIASLTFLYTLLREVIHPLATSHQQ
YFYGGGGSGGGGSGGGGSLVYLPGVIAAIVQLLTRKQFGLLSFFFAVLHA
IYSLSYPGGGGSGGGGSGGGGSSQSTIPIVGIVAGLAVLAVVVIGAVVAT
VMCRRKSSGGKGGSYSQAASSDSAQGSDVSLTAGGGGSGGGGSGGGGSIV
GIVAGLAVLAVLAVLGAMVAVVMCRRKSSGGKGGSCSQAASSNSAQGSDV
SLTA.

The amino acid sequence of mRNA-3 was as follows:

(SEQ ID NO: 3)
MRVTAPRTVLLLLSAALALTETWAAKFVAAWTLKAAAMWNLLHETDSAVA
TARRPRWVLAGGFFLLKKFLYNFTQIPHLAGTGGGGSGGGGSGGGGSNVS
DIVPPFSAFSPQGMPEGDLVYVNYARTEDFFKLEGGGGSGGGGSGGGGSA
RYGKVFRGNKVKNAQLAGAKGVILYSDPADYFAPGGGGGSGGGGSGGGGS
SRLLQERGVAYINADSSIEGNYTLRVDCTPLMYSLVHNLTKEGGGGSGGG
GSGGGGSFYDPMFKYHLVLRKYADKIYSISMKHPQEMKTYSVSFDSLFSA
VGGGGSGGGGSGGGGSVLRMMNDQLMFLSHNKYAGESFPGIYDALFDIES
KVDPSGGGGSGGGGSGGGGSAGLALQPGTALLCYSNCTQLGEQCWTARIR
GGGGSGGGGSGGGGSDTDLCNASGAHALQPAAAILALLPALGLLLWGPGQ
LGGGGSGGGGSGGGGSPQWHLPIKIAAIIASLTFLYTLLREVIHPLATSH
QQYFYGGGGSGGGGSGGGGSLVYLPGVIAAIVQLLTRKQFGLLSFFFAVL
HAIYSLSYPGGGGSGGGGSGGGGSSQSTIPIVGIVAGLAVLAVVVIGAVV
ATVMCRRKSSGGKGGSYSQAASSDSAQGSDVSLTAGSGATNFSLLKQAGD
VEENPGPMYRMQLLSCIALSLALVTNSAPTSSSTKKTQLQLEHLLLDLQM
ILNGINNYKNPKLTRmLTFKFYMPKKATELKHLQCLEEELKPLEEVLNLA
QSKNFHLRPRDLISNINVIVLELKGSETTFMCEYADETATIVEFLNRWIT
FCQSIISTLT.

The preparation of the mRNA-1, mRNA-2, and mRNA-3 was conducted as follows:

### S1: Plasmid Digestion and Linearized Template Preparation

S1: Components listed in Table 1 for plasmid linearization were thawed on ice. Based on a 40 μg plasmid linearization system, the required amounts of each component were calculated. The components were sequentially added to 1.5 mL microcentrifuge tubes and thoroughly mixed to prepare reaction solutions 01, 02, and 03 (corresponding to reaction mixtures containing plasmids SJ222-01, SJ222-02, and SJ222-03, respectively).

TABLE 1
Plasmid linearization reaction system (400 μL)

| Plasmid name | Reagent name | Volume (μL) |
|---|---|---|
| SJ222-01 | pDNA (2.9 mg/mL) | 14 |
| | Restriction enzyme buffer (10X) | 40 |
| | BspQI | 8 |
| | Water for injection (WFI) | 338 |
| | Total | 400 |
| SJ222-02 | pDNA (1.3 mg/mL) | 31 |
| | Restriction enzyme buffer (10X) | 40 |
| | BspQI | 8 |
| | WFI | 321 |
| | Total | 400 |
| SJ222-03 | pDNA (2.7 mg/mL) | 15 |
| | Restriction enzyme buffer (10X) | 40 |
| | BspQI | 8 |
| | WFI | 337 |
| | Total | 400 |

S1.2: The mixed reaction solutions 01, 02, and 03 were placed in an oscillation reactor with parameters set to 50° C., 300 rpm, for a reaction duration of 2 h.
S1.3: After completion of the reaction, the linearized plasmid reaction mixtures were purified. For each tube, 400 μL of DNA magnetic beads were added for purification, followed by elution with 100 μL of WFI to collect the samples. This yielded purified linearized plasmids SJ222-01, SJ222-02, and SJ222-03, respectively.
   S2: In Vitro Transcription (IVT) for mRNA Preparation

S2.1: Components of the IVT reaction system listed in Table 2 were thawed on ice. Based on a 20 μg IVT system, the required amounts of each component were calculated. The components were sequentially added to 1.5 mL microcentrifuge tubes and thoroughly mixed to prepare reaction solutions a, b, and c (corresponding to reaction mixtures containing linearized plasmids SJ222-01, SJ222-02, and SJ222-03, respectively).

TABLE 2
IVT reaction system

| Reagent name | Volume (μL) |
|---|---|
| Lin-pDNA (0.8 mg/mL) | 25 |
| T7 reaction buffer | 40 |
| ATP | 40 |
| GTP | 40 |
| CTP | 40 |
| N1-Me-pUTP | 40 |
| CAP analog | 40 |
| T7 RNA polymerase | 20 |
| Inorganic pyrophosphatase | 0.8 |
| RNase inhibitor | 20 |
| WFI | 95 |
| Total | 400.8 |

In Table 2, “Lin-pDNA” referred to the linearized plasmids SJ222-01, SJ222-02, or SJ222-03.

S2.2: The blended reaction solutions a, b, and c were placed in a oscillation reactor with parameters set to 37° C., 300 rpm, for a reaction duration of 2.5 h. After the reaction, 40 μL of DNase I was added to each tube, mixed thoroughly. Then the reaction was conducted at 37° C. for 15 min, yielding IVT reaction solutions a, b, and c.
S2.3: After completion of the reaction, IVT reaction solutions a, b, and c were purified by adding 800 μL of RNA magnetic beads to each tube. The samples were eluted with 1 mL of WFI to obtain the stock solutions, designated as mRNA-1, mRNA-2, and mRNA-3.
S2.4: The concentrations of mRNA-1, mRNA-2, and mRNA-3 were measured and adjusted to 1.0 mg/mL using WFI. Total amount and yield were calculated, and integrity, capping efficiency, and Poly(A) were assessed.

Sequencing confirmed the successful preparation of: mRNA-1 having the amino acid sequence set forth in SEQ ID NO: 1, mRNA-2 having the amino acid sequence set forth in SEQ ID NO: 2, and mRNA-3 having the amino acid sequence set forth in SEQ ID NO: 3.

### Capping Efficiency Analysis Protocol:

(1) Reaction Setup:

The mRNA to be tested and corresponding probes were thawed on ice, vortex-mixed, and briefly centrifuged. The enzymatic digestion system was prepared as outlined in the table below.

TABLE 3
Enzyme digestion system

| Component | Volume |
|---|---|
| mRNA to be tested | 100 pmole |
| 10× reaction buffer | 12 μL |
| Biotin-labeled probe | 100 pmole |
| Nuclease-free water | supplemented to 120 μL |

(2) Annealing: annealing was conducted in a PCR thermocycler under the following conditions: 95° C. for 5 min, 65° C. for 2 min, 55° C. for 2 min, 40° C. for 2 min, and 22° C. for 2 min. [0082](3) Magnetic Bead Pre-Washing: for each sample, 100 μL of streptavidin (SA) beads was transferred to a tube. The beads were immobilized on a magnetic stand, and the supernatant was removed. The beads were washed sequentially with 100 μL of Wash Buffer (3 times) and 100 μL of Solution A (2 times), then resuspended in 100 μL of Solution B for later use.
(4) Bead Binding: after immobilizing the beads and discarding the supernatant, the annealed products were added to resuspend the beads, and placed at room temperature for 30 min.
(5) A total of 5 μL of RNase H was added to each sample. The mixture was vortexed, centrifuged, and incubated at 37° C. in a metal bath with shaking at 1,200 rpm for 2 h.
(6) The beads were immobilized, and the supernatant was removed. The beads were washed with Wash Buffer (3 times) and DEPC-treated water (3 times). Subsequently, 100 μL of 75% methanol was added to resuspend the beads, which were heated at 80° C. in a metal bath for 3 min. The supernatant was collected after bead immobilization while it was hot.
(7) The supernatant was evaporated to dryness using a rotary evaporator at room temperature. The resulting product was reconstituted in 25 μL of blank solution or DEPC-treated water, immobilized on a magnetic stand, and the supernatant was collected for concentration measurement.
(8) Samples were either analyzed or stored at −80° C. for later use.

### Poly(A) Tail Analysis Protocol:

(i) Enzymatic Digestion: 100 μg of mRNA sample was aliquoted using a pipette and added 10 μL of RNase T1 and 10× RNase H reaction buffer (final concentration 1×). The mixture was mixed thoroughly and incubated at 37° C. in a thermostatic oscillator for 0.5 h.
(ii) 500 μL of magnetic beads was transferred to a centrifuge tube. The beads were immobilized on a magnetic stand, and the supernatant was removed. The beads were resuspended in 100 μL of Binding Buffer, followed by addition of the digested sample. After thorough mixing via pipetting, the mixture was gently agitated on an oscillator and incubated at room temperature for 10 min to ensure Poly(A) tail binding to the beads.
(iii) After incubation, the beads were immobilized, and the supernatant was discarded. The beads were washed with Wash Buffer (3 times) and 100 mM ammonium acetate solution (3 times). Subsequently, 100 μL of 75% methanol was added to resuspend the beads, which were heated at 80° C. in a metal bath for 3 min. The supernatant was collected after bead immobilization while it was hot.
(iv) The supernatant was evaporated to dryness using a rotary evaporator at room temperature. The resulting product was reconstituted in 25 μL of blank solution or DEPC-treated water, immobilized on a magnetic stand, and the supernatant was collected for concentration measurement.
(v) Samples were either analyzed or stored at −80° C. for later use.

The test results were shown in Table 4.

TABLE 4
Results of concentration, total amount, yield, integrity,
capping efficiency, and poly (A) of different mRNA samples

| | | | Total | | | | |
|---|---|---|---|---|---|---|---|
| mRNA | Concentration | Volume | amount | | | Capping | |
| name | (ng/μL) | (mL) | (mg) | Yield | Integrity | efficiency | Poly(A) |
| mRNA-1 | 1053 | 3.6 | 3.7 | 189 | 94.4% | 96.3% | 31-33A; |
| | | | | folds | | | 71-85A |
| mRNA-2 | 1064.8 | 3.6 | 3.7 | 186 | 92.1% | 96.6% | 30-34A; |
| | | | | folds | (shoulder | | 71-82A |
| | | | | | peak) | | |
| mRNA-3 | 1071.8 | 3.6 | 3.7 | 192 | 91.10% | 96.7% | 31-33A; |
| | | | | folds | | | 71-85A |

As shown in Table 4, the three prepared mRNA samples exhibited high concentrations, all exceeding 1,050 ng/μL, making them suitable for subsequent LNP encapsulation and applications. The production yields of the mRNA samples ranged between 186- and 192-fold, demonstrating high efficiency, low variability, and a robust, reliable methodology. The integrity of the mRNA samples was 91.1% to 94.4%, indicating minimal damage and degradation during preparation and preservation of full-length mRNA structures, which was critical for functional studies and applications. The capping efficiency of the mRNA samples was 96.3% to 96.7%, the high capping efficiency is essential for enhancing mRNA stability and translation efficiency. The Poly(A) tail lengths were distributed within 31-33A or 30-34A (short-chain) and 71-85A or 71-82A (long-chain), suggesting uniform distribution of Poly(A) lengths that further contributed to enhanced mRNA stability and translation efficiency.

### Comparative Example 1

Preparation Method of an mRNA Vaccine

### (1) Aqueous Phase Solution

The mRNA-1 prepared in Example 1 was diluted with WFI to 183.5 μg/mL, followed by further dilution to 91.75 μg/mL using a 100 mM citrate buffer (pH=4.0), yielding the aqueous phase solution.

### (2) Lipid Phase Solution

A lipid phase solution was prepared at a molar ratio of SM-102:DSPC:cholesterol:DMG-PEG2000=50:10:38.5:1.5. The final concentrations were: SM-102 (5.00 mM), DSPC (1.00 mM), cholesterol (3.85 mM), and DMG-PEG2000 (0.15 mM).

### (3) Combining of Aqueous and Lipid Phases

Using a microfluidic device, 9 mL of the aqueous phase solution and 3 mL of the lipid phase solution were mixed under the following parameters: N/P ratio (molar ratio of nitrogen to phosphorus is molar ratio of nitrogen to phosphorus atoms)=6:1, aqueous phase flow rate: ethanol phase flow rate=3:1, and total flow rate=12 mL/min. A resulting mRNA vaccine was obtained and designated as mRNA LNP-1.

### (4) Buffer Exchange

The mRNA vaccine from step (3) underwent buffer exchange via ultrafiltration into a storage solution.

Preparation of storage solution: a solution containing 10 mM Tris buffer (1.21 g), 0.1 g NaCl, and 80 g sucrose was dissolved in ddH₂O to a final volume of 1 L, adjusted to a pH value of 7.4, and thoroughly mixed.

### Example 2

Preparation Method of an mRNA Vaccine

### (1) Aqueous Phase Solution

The mRNA-2 prepared in Example 1 was diluted with WFI to 183.5 μg/mL, followed by further dilution to 91.75 μg/mL using a 100 mM citrate buffer (Cit, pH=4.0), yielding the aqueous phase solution.

### (2) Lipid Phase Solution

A lipid phase solution was prepared at a molar ratio of SM-102:DSPC:cholesterol:DMG-PEG2000=50:10:38.5:1.5. The final concentrations were: SM-102 (5.00 mM), DSPC (1.00 mM), cholesterol (3.85 mM), and DMG-PEG2000 (0.15 mM).

### (3) Combining of Aqueous and Lipid Phases

Using a microfluidic device, 9 mL of the aqueous phase solution and 3 mL of the lipid phase solution were mixed under the following parameters: N/P ratio (molar ratio of nitrogen to phosphorus)=6:1, aqueous phase flow rate: ethanol phase flow rate=3:1, and total flow rate=12 mL/min. A resulting mRNA vaccine was obtained and designated as mRNA LNP-2.

### (4) Buffer Exchange

The mRNA vaccine from step (3) underwent buffer exchange via ultrafiltration into a storage solution.

Preparation of storage solution: a solution containing 10 mM Tris buffer (1.21 g), 0.1 g NaCl, and 80 g sucrose was dissolved in ddH₂O to a final volume of 1 L, adjusted to a pH value of 7.4, and thoroughly mixed.

### Comparative Example 2

Preparation Method of an mRNA Vaccine

### (1) Aqueous Phase Solution

The mRNA-3 prepared in Example 1 was diluted with WFI to 183.5 μg/mL, followed by further dilution to 91.75 μg/mL using a 100 mM citrate buffer (pH=4.0), yielding the aqueous phase solution.

### (2) Lipid Phase Solution

A lipid phase solution was prepared at a molar ratio of SM-102:DSPC:cholesterol:DMG-PEG2000=50:10:38.5:1.5. The final concentrations were: SM-102 (5.00 mM), DSPC (1.00 mM), cholesterol (3.85 mM), and DMG-PEG2000 (0.15 mM).

### (3) Combining of Aqueous and Lipid Phases

Using a microfluidic device, 9 mL of the aqueous phase solution and 3 mL of the lipid phase solution were mixed under the following parameters: N/P ratio (molar ratio of nitrogen to phosphorus)=6:1, aqueous phase flow rate: ethanol phase flow rate=3:1, and total flow rate=12 mL/min. A resulting mRNA vaccine was obtained and designated as mRNA LNP-3.

### (4) Buffer Exchange

The mRNA vaccine from step (3) underwent buffer exchange via ultrafiltration into a storage solution.

Preparation of storage solution: a solution containing 10 mM Tris buffer (1.21 g), 0.1 g NaCl, and 80 g sucrose was dissolved in ddH₂O to a final volume of 1 L, adjusted to a pH value of 7.4, and thoroughly mixed.

### Example 3

I. The particle size, polydispersity index (PDI), encapsulation efficiency, total mRNA concentration, and free mRNA concentration of mRNA LNP-1 (prepared in Comparative Example 1), mRNA LNP-2 (prepared in Example 2), and mRNA LNP-3 (prepared in Comparative Example 2) were determined, with results summarized in Table 5.

TABLE 5
Characterization results of different mRNA vaccines

| | Particle | | Encapsula- | Total mRNA | Free mRNA |
|---|---|---|---|---|---|
| Sample | size | | tion | concentration | concentration |
| Name | (nm) | PDI | rate (%) | (μg/mL) | (μg/mL) |
| mRNA | 98.14 | 0.044 | 96.28 | 144.52 | 5.38 |
| LNP-1 | | | | | |
| mRNA | 90.63 | 0.056 | 93.28 | 140.17 | 9.42 |
| LNP-2 | | | | | |
| mRNA | 91.34 | 0.040 | 95.4 | 137.05 | 6.3 |
| LNP-3 | | | | | |

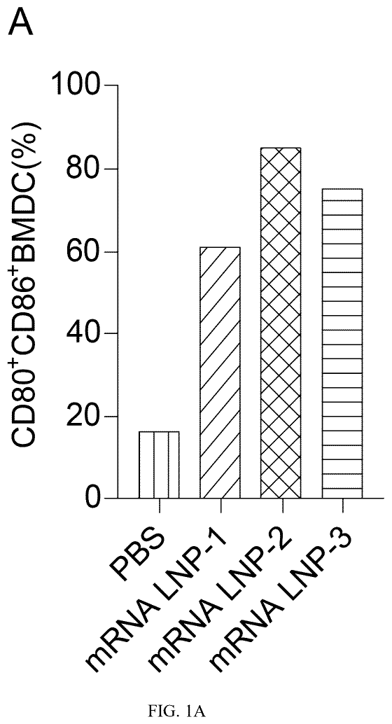

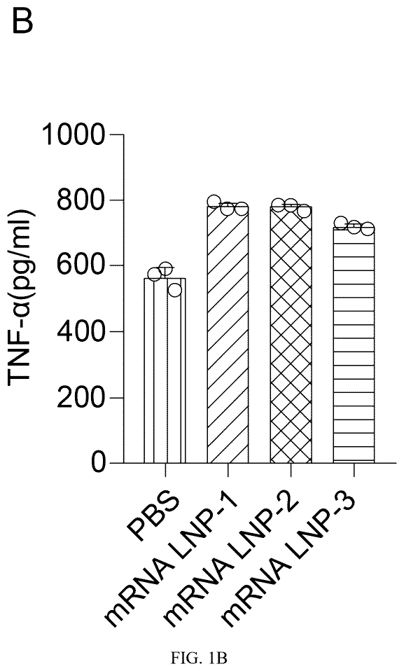

The results in Table 5 showed that the preparation method of the present disclosure could successfully obtain nano-scale mRNA vaccines with high encapsulation rates of 96.28%, 93.28% and 95.4%, respectively.
II. Immune Response Assessment of mRNA LNP-1 (prepared in Comparative Example 1), mRNA LNP-2 (prepared in Example 2), and mRNA LNP-3 (prepared in Comparative Example 2)
(1) BMDCs from C57BL/6 mice were added to 6-well plates at 1×10⁶ cells per well. The cells were treated with BMDC-containing medium including 10 μg/mL mRNA LNP-1, 10 μg/mL mRNA LNP-2, 10 μg/mL mRNA LNP-3, and PBS (control) in for 24 h, respectively. The expression of co-stimulatory molecules CD80 and CD86 on surface of BMDCs was analyzed by flow cytometry (FIG. 1A). The content of TNF-α secreted by BMDCs was detected via ELISA (FIG. 1B).

Results from FIGS. 1A-1B revealed that the percentages of CD80⁺CD86⁺ BMDCs stimulated by PBS, mRNA LNP-1, mRNA LNP-2, and mRNA LNP-3 were 16.2%, 60.9%, 84.7%, and 74.9%, respectively. Content of TNF-α secreted by stimulated BMDCs were 565.0 pg/mL (PBS), 781.1 pg/mL (mRNA LNP-1), 777.3 pg/mL (mRNA LNP-2), and 718.4 pg/mL (mRNA LNP-3), respectively. The mRNA LNP-2 exhibited the strongest activity in promoting BMDC maturation and pro-inflammatory cytokine secretion.
(2) The BMDCs stimulated in step (1) were co-incubated with T lymphocytes in spleen of C57BL/6 mouse at a cell ratio of 1:10 for 72 h. A resulting T lymphocytes in spleen of mouse were then co-cultured with Tramp C1, a murine prostate cancer cell line expressing PSMA, PSCA, and STEAP1 (purchased from ATCC, USA), at a cell ratio of 20:1 for 24 h. Content of Lactate dehydrogenase (LDH) in a resulting supernatant were measured and the killing efficiency of T lymphocytes induced by different treatment groups against Tramp C1 was calculated. Content of LDH was measured using an LDH Cytotoxicity Assay Kit (purchased from Beyotime Biotechnology, Catalog No. C0017). The killing efficiency (%) was calculated as follows: cell killing efficiency (%)=(target cell-effector cell mixed OD value-effector cell-control well OD value-target cell-control well OD value)÷(target cell maximum enzyme activity-control well OD value-target cell-control well OD value)×100%.

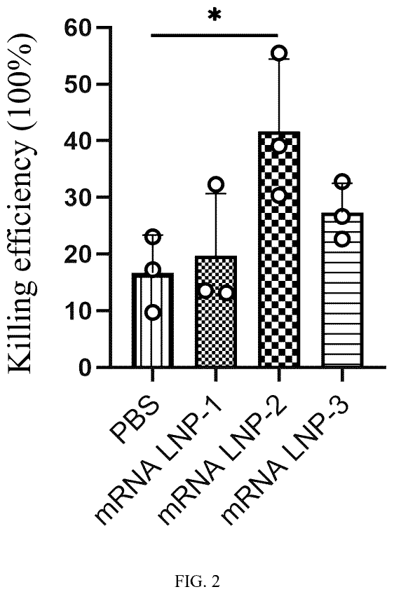

FIG. 2 showed the results that the killing efficiencies of PBS, mRNA LNP-1, mRNA LNP-2, and mRNA LNP-3 induced T lymphocytes against Tramp C1 cells (at a 20:1 E:T ratio) were 16.7%, 19.7%, 41.7%, and 27.4%, respectively. The effector T cells induced by mRNA LNP-2 showed the highest killing efficiency of against target cells (prostate cancer cells).
(3) Induction of Human Monocyte-Derived Dendritic Cells (MoDCs):
A1. Fresh human platelet-rich buffy coat (collected from 400 mL of whole blood, volume: about 40-45 mL) was subjected to PBMC isolation via Ficoll density gradient centrifugation. Briefly, 5 mL of buffy coat was layered over 5 mL of Ficoll solution in a 15 mL centrifuge tube and centrifuged at 700×g for 25 min. The mononuclear cell layer was carefully aspirated and washed twice with PBS.
A2. The isolated PBMCs were dispersed in 1640 medium supplemented with 1% fetal bovine serum (FBS) and transferred to culture flasks for adherent cell enrichment for 2 hours.
A3. The culture flasks was gentle agitated and non-adherent cells were removed (human peripheral blood T lymphocytes), the remaining adherent cells were cultured in 1640 complete medium containing 10% FBS, 200 ng/mL GM-CSF, and 100 ng/mL IL-4 for 6-8 d. Half-medium were changes to suppliment fresh cytokines every other day.
A4. MoDCs were inoculated into 6-well plates at 3×10⁶ cells in 2 mL of medium per well. The cytokine in medium was maintained.

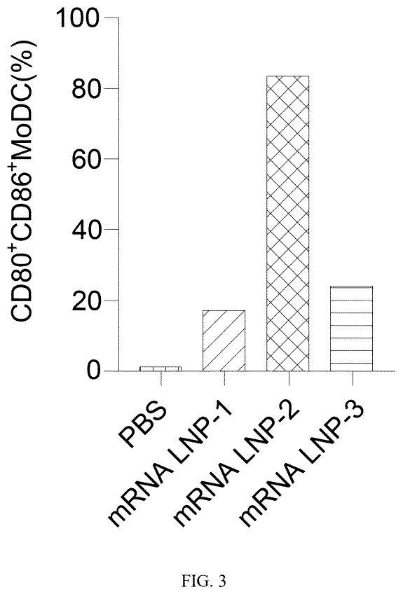

1×10⁶ MoDCs were added to each well of the 6-well plate. The cells were stimulated with MoDC medium supplemented with 10 μg/mL mRNA LNP-1, 10 μg/mL mRNA LNP-2, 10 μg/mL mRNA LNP-3, and PBS (control) for 24 h, respectively. Expression of costimulatory molecules CD80 and CD86 on surface of MoDCs was analyzed by flow cytometry (FIG. 3).

FIG. 3 showed the results that the percentages of CD80⁺CD86⁺ MoDCs stimulated by PBS, mRNA LNP-1, mRNA LNP-2, and mRNA LNP-3 were 1.14%, 17.2%, 83.5%, and 24%, respectively.

### Example 4

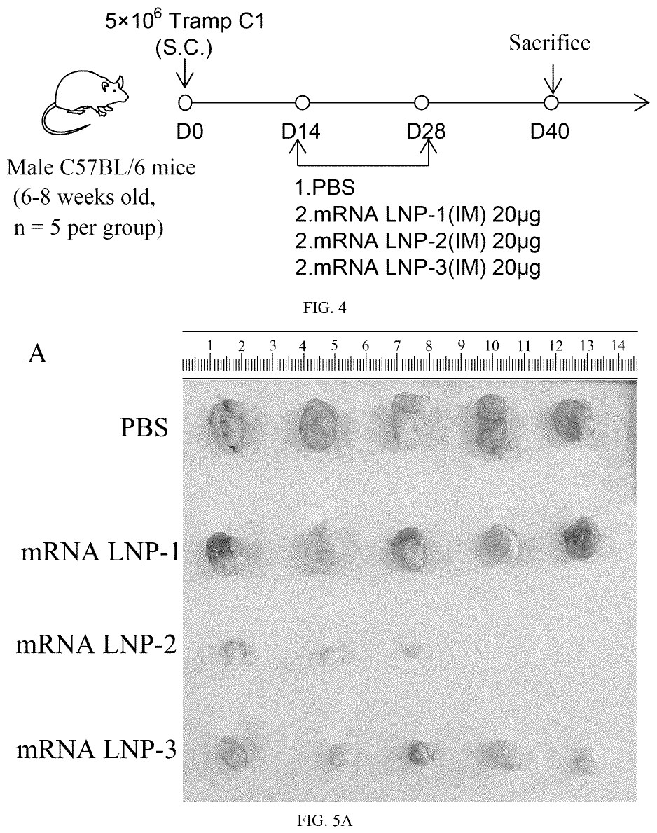

Therapeutic Efficacy in Subcutaneous Tumor-Bearing Mouse Model: the animal model selection and treatment timeline are illustrated in FIG. 4. Detailed procedures were as follows:

The mRNA vaccines used in this example included mRNA LNP-1 (prepared by Comparative Example 1), mRNA LNP-2 (prepared by Example 2), and mRNA LNP-3 (prepared by Comparative Example 2).

Treatment Protocol for Subcutaneous tumor model of Prostate Cancer in mice: 20 male C57BL/6 mice (6-8 weeks old, purchased from SPF (Beijing) Biotechnology Co., Ltd.) were divided into 4 groups (n=5 per group). Each group received intramuscular injections of PBS (140 μL), mRNA LNP-1 (20 μg), mRNA LNP-2 (20 g), and mRNA LNP-3 (20 μg), respectively. Tramp C1 cells (3×10⁶ cells/mouse) were subcutaneously implanted into the left inguinal region on day 0. mRNA vaccines or PBS were intramuscular injected on days 14 and 28. Body weight and tumor volume were recorded every 5 d. On day 40, mice were euthanized, and tumor size/weight of day 40 were measured. Spleens and lymph nodes were harvested for immunological analyses, including immunohistochemistry, flow cytometry, ELISpot, and LDH assays.

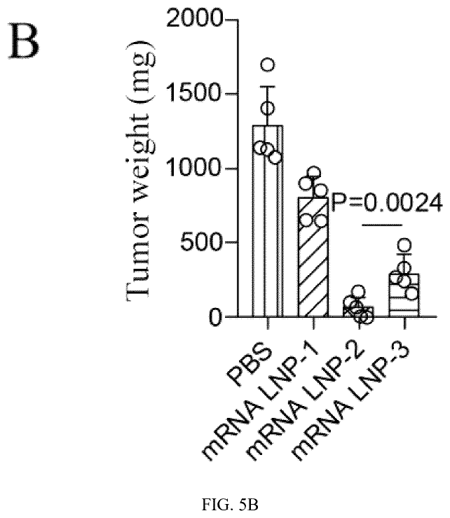

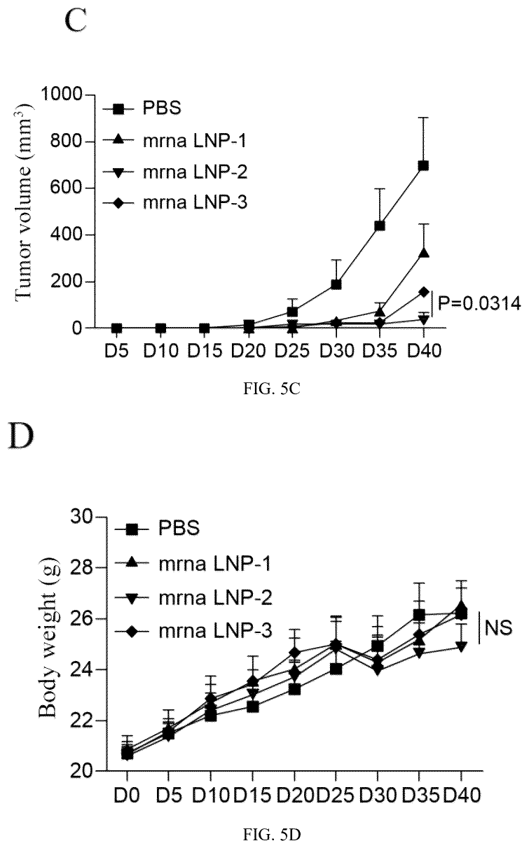

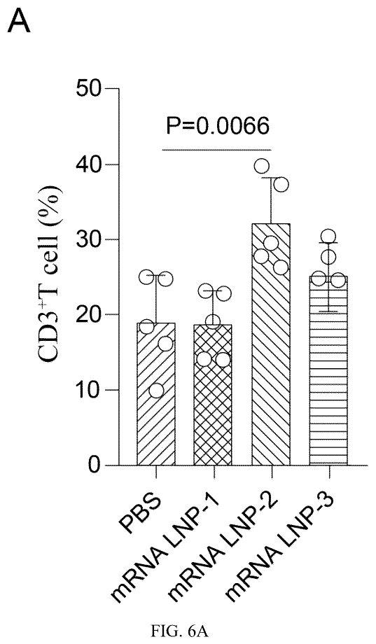

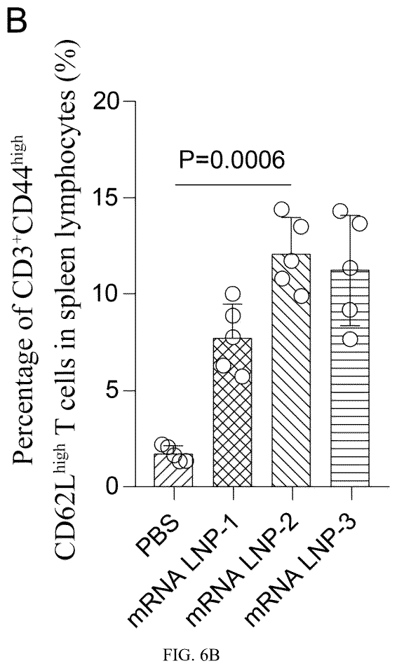

FIGS. 5A-5D showed results that mRNA LNP-2 exhibited the most potent tumor-suppressive effect, with the lowest tumor volume and mass in mRNA LNP-2 group. No significant differences in body weight were observed between groups. FIGS. 6A-6B showed results that mRNA LNP-2 group showed the highest proportions of infiltrating T lymphocytes and central memory T lymphocytes in the spleen.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A messenger ribonucleic acid (mRNA) having the amino acid sequence set forth in SEQ ID NO: 2.

2. An mRNA vaccine formulation, comprising the mRNA according to claim 1.

3. The mRNA vaccine formulation according to claim 2, wherein a carrier for the mRNA vaccine formulation is a lipid nanoparticle (LNP).

4. A preparation method of an mRNA vaccine formulation, comprising the following steps:
diluting the mRNA according to claim 1 to obtain an aqueous phase solution;
mixing 9-heptadecanyl 8-{(2-hydroxyethyl)[6-oxo-6-(undecyloxy)hexyl]amino}octanoate (SM-102), 1,2-distearoyl-sn-glycero-3-phosphocholine (DSPC), cholesterol, and 1,2-dimyristoyl-rac-glycero-3-methoxypolyethylene glycol 2000 (DMG-PEG2000) to obtain a lipid phase solution; and
combining the aqueous phase solution and the lipid phase solution to obtain the mRNA vaccine formulation.

5. The preparation method according to claim 4, wherein the mRNA is diluted to a concentration of 90 μg/mL to 95 μg/mL; the SM-102, the DSPC, the cholesterol, and the DMG-PEG2000 are mixed at a molar ratio of (45-55):(9-11):(35-40):(1-2); and the aqueous phase solution and the lipid phase solution are combined at a volume ratio of (1-6):(1-3).

6. A use of the mRNA according to claim 1 in preparing a dendritic cell, wherein the dendritic cell is mature and/or secretes a pro-inflammatory cytokine.

7. A use of the mRNA according to claim 1 in preparing a drug for treating a prostate cancer.

8. A method for preparing a dendritic cell, wherein the dendritic cell is mature and/or secretes a pro-inflammatory cytokine, comprising co-incubating a native dendritic cell with the mRNA vaccine formulation according to claim 2.

9. A method for preparing an effector T cell, comprising co-incubating the dendritic cell prepared by the method according to claim 8 with a T lymphocyte.

10. A use of the effector T cell prepared by the method according to claim 9 in preparing a drug for treating a prostate cancer.

11. A use of the mRNA vaccine formulation according to claim 2 in preparing a dendritic cell, wherein the dendritic cell is mature and/or secretes a pro-inflammatory cytokine.

12. The use according to claim 11, wherein a carrier for the mRNA vaccine formulation is an LNP.

13. A use of the mRNA vaccine formulation prepared by the preparation method according to claim 4 in preparing a dendritic cell, wherein the dendritic cell is mature and/or secretes a pro-inflammatory cytokine.

14. A use of the mRNA vaccine formulation according to claim 2 in preparing a drug for treating a prostate cancer.

15. The use according to claim 14, wherein a carrier for the mRNA vaccine formulation is an LNP.

16. A use of the mRNA vaccine formulation prepared by the preparation method according to claim 4 in preparing a drug for treating a prostate cancer.

17. The method according to claim 8, wherein a carrier for the mRNA vaccine formulation is an LNP.

18. A method for preparing a dendritic cell, wherein the dendritic cell is mature and/or secretes a pro-inflammatory cytokine, comprising co-incubating a native dendritic cell with the mRNA vaccine formulation according to the mRNA vaccine formulation prepared by the preparation method according to claim 4.

19. A method for preparing an effector T cell, comprising co-incubating the dendritic cell prepared by the method according to claim 18 with a T lymphocyte.

20. A use of the effector T cell prepared by the method according to claim 19 in preparing a drug for treating a prostate cancer.
